(19) United States
(12) Patent Application Publication
(10) Pub. No.: US 2026/0364100 A1
(43) Pub. Date: Nov. 19, 2026

(54) COMPOSITION FOR THE PREVENTION OR TREATMENT OF NEUROFIBROMATOSIS TYPE 1

(71) Applicant: PUSAN NATIONAL UNIVERSITY INDUSTRY-UNIVERSITY COOPERATION FOUNDATION, Geumjeong-gu (KR)

(72) Inventors: Su Jung PARK, Yangsan-si (KR); Han Sol JUNG, Yangsan-si (KR); Ji-Hee KIM, Yangsan-si (KR); Chong Kun CHEON, Yangsan-si (KR)

(21) Appl. No.: 19/661,969

(22) Filed: Apr. 29, 2026

(30) Foreign Application Priority Data
May 9, 2025 (KR) 10-2025-0060120

(51) Int. Cl. A61K 31/704; A61K 31/7048; A61P 35/00

(52) U.S. Cl. CPC A61K 31/704; A61K 31/7048; A61P 35/00

(57) ABSTRACT

The present invention relates to a composition for the treatment of neurofibromatosis type 1, comprising Raddeanin A or Polyphyllin B as an active ingredient. The composition of the present invention inhibits FBXW11, which induces the degradation of neurofibromin (NF1), and thereby induces an increase in NF1 protein through the inhibition of the interaction between FBXW11 and NF1; thus, it can be usefully used for the treatment of neurofibromatosis type 1.

### CROSS-REFERENCE TO RELATED PATENT APPLICATION

This application claims the benefit of Korean Patent Application No. 10-2025-0060120, filed on May 9, 2025, in the Ministry of Intellectual Property (MOIP), the disclosure of which is incorporated herein in its entirety by reference.

### BACKGROUND OF THE INVENTION

### 1. Field of the Invention The present invention relates to a composition for the prevention or treatment of neurofibromatosis type 1, comprising Raddeanin A or Polyphyllin B as an active ingredient.

### 2. Description of the Related Art

Neurofibromatosis type 1 (NF1) is a rare genetic disease that manifests in various ways across the central and peripheral nervous systems, as well as the skeletal system, soft tissues, and skin, and is known to affect approximately 1 in 3,000 individuals.

Approximately 50% of cases are inherited, while the remaining 50% result from spontaneous de novo mutations, and generally, adults with NF1 develop an increasing number of neurofibromas over time. Even among NF1 patients within the same family who share the same genotype, the phenotype can vary significantly, and the characteristic process of tumorigenesis and malignant transformation is a hallmark of NF1.

Symptoms of NF1 occur wherever nerves are located, and in addition to manifestations such as cutaneous neurofibromas, cafe-au-lait spots, axillary freckling, inguinal freckling, Lisch nodules, optic gliomas, scoliosis, tibial dysplasia, plexiform neurofibromas, and pontine gliomas, they include neurocognitive manifestations such as short stature and cognitive deficits, including learning disabilities.

NF1 is caused by mutations in the NF1 gene. The NF1 gene is located on the long arm of chromosome 17 (17q11.2) and encodes a 280-320 kDa cytoplasmic GTPase-activating protein (GAP) called neurofibromin. Neurofibromin binds to GTP-RAS and functions to stimulate the hydrolysis of GTP bound to the oncogenic RAS, thereby regulating RAS activity by converting RAS-GTP into RAS-GDP. When the ability to control RAS activation is lost due to haploinsufficiency caused by mutations in the NF1 allele, the RAS protein becomes hyperactivated and GTP-RAS accumulates, leading to increased cell proliferation and the development of NF1.

Therapeutic techniques for NF1 primarily involve surgical intervention; however, surgery is often impossible, creating a demand for the development of therapeutic agents. In 2020, selumetinib was approved by the U.S. FDA as the first treatment for neurofibromatosis type 1. Selumetinib (AstraZeneca) is a drug that targets MEK in the RAS-MAPK pathway; however, it is currently indicated only for the treatment of plexiform neurofibromas, which limits its application compared to the diverse symptoms of NF1. Furthermore, there is a high possibility of recurrence upon discontinuation of the drug, and issues regarding drug resistance have recently been reported.

In addition, most research on the development of NF1 therapeutics targets the RAS signaling pathway, and there is a critical need for the development of novel therapeutic agents due to the limitations of existing treatments compared to the diverse symptoms of NF1, including cutaneous neurofibromas and neurocognitive and behavioral deficits in children.

### SUMMARY OF THE INVENTION

Based on the aforementioned problems associated with the prior art, the inventors of the present invention have discovered FBXW11 as a therapeutic target gene involved in the intracellular degradation of neurofibromin, in order to overcome the symptoms of neurofibromatosis type 1 (haploinsufficiency) using drugs that inhibit the degradation of neurofibromin within cells. Moreover, the efficacy of FBXW11 as a therapeutic target gene was demonstrated using an Nf1^{+/-} cognitive impairment model, and Raddeanin A and Polyphyllin B were discovered through inhibitor screening to suppress the interaction between FBXW11 and neurofibromin, thereby completing the present invention.

Accordingly, an object of the present invention is to provide a pharmaceutical composition for the prevention or treatment of neurofibromatosis type 1, comprising Raddeanin A, Polyphyllin B, or a pharmaceutically acceptable salt thereof as an active ingredient.

Another object of the present invention is to provide a method for treating neurofibromatosis type 1, comprising administering Raddeanin A, Polyphyllin B, or a pharmaceutically acceptable salt thereof to a subject.

Still another object of the present invention is to provide a food composition for the prevention or improvement of neurofibromatosis type 1, comprising Raddeanin A, Polyphyllin B, or a pharmaceutically acceptable salt thereof as an active ingredient.

The terms used in this specification are intended only for the purpose of illustration and should not be construed as limiting. Unless the context clearly indicates otherwise, the singular forms also include the plural forms. It should be understood that, as used herein, the terms “comprise”, “have”, or the like are intended to specify the presence of stated features, numerals, steps, operations, components, parts, or combinations thereof, but do not preclude the presence or addition of one or more other features, numerals, steps, operations, components, parts, or combinations thereof.

Unless otherwise defined, all terms used herein, including technical and scientific terms, have the same meaning as commonly understood by those skilled in the art to which the embodiments pertain. Terms, such as those defined in commonly used dictionaries, should be interpreted as having a meaning that is consistent with their meaning in the context of the relevant art, and will not be interpretedd in an idealized or overly formal sense unless explicitly defined otherwise.

Hereinafter, the present invention will be described in detail.

To achieve the above objects, one aspect of the present invention provides a pharmaceutical composition for the prevention or treatment of neurofibromatosis type 1, comprising Raddeanin A, Polyphyllin B, or a pharmaceutically acceptable salt thereof as an active ingredient.

The “Raddeanin A” used in the present invention is an oleanane-type triterpenoid saponin compound primarily isolated from the rhizomes of Anemone raddeana. Structurally, it is in the form of a glycoside in which several sugar molecules are attached to a triterpene skeleton, and is known as a natural compound having effects on rheumatism, arthritis, and pain relief. However, there has been no report on the use of Raddeanin A for the prevention and treatment of neurofibromatosis type 1 through the inhibition of intracellular NF1-FBXW11 binding and the increase in NF1 protein.

The “Polyphyllin B” used in the present invention is a steroidal saponin-based compound primarily isolated from the rhizome of Paris polyphylla, also known as Chilyeobiljihwa or Jungru. It has a steroid glycoside structure with pennogenin as an aglycone, and is known as a natural compound effective for detoxification, anti-inflammation, and hemostasis. However, there has been no report on the use of Polyphyllin B for the prevention and treatment of neurofibromatosis type 1 through the inhibition of intracellular NF1-FBXW11 binding and the increase in NF1 protein.

The compounds of the present invention may be used in the form of a pharmaceutically acceptable salt, and include all salts, hydrates, and solvates prepared by conventional methods.

As the salt, acid addition salts formed by pharmaceutically acceptable free acids are useful. The acid addition salts are prepared by conventional methods, for example, by dissolving a compound in an excess amount of an aqueous acid solution and precipitating the salt using a water-miscible organic solvent, such as methanol, ethanol, acetone, or acetonitrile. Equimolar amounts of a compound and an acid in water or an alcohol (e.g., glycol monomethyl ether) may be heated, and then the mixture may be evaporated to dryness, or the precipitated salt may be filtered by suction. In this case, organic acids and inorganic acids may be used as the free acids; hydrochloric acid, phosphoric acid, sulfuric acid, nitric acid, tartaric acid, and the like may be used as the inorganic acids; methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycollic acid, gluconic acid, galacturonic acid, glutamic acid, glutaric acid, glucuronic acid, aspartic acid, ascorbic acid, carbonic acid, vanillic acid, hydroiodic acid, and the like may be used as the organic acids, but are not limited thereto.

The terms used in this specification are intended only for the purpose of illustration and should not be construed as limiting. Unless the context clearly indicates otherwise, the singular forms also include the plural forms. It should be understood that, as used herein, the terms “comprise”, “have”, or the like are intended to specify the presence of stated features, numerals, steps, operations, components, parts, or combinations thereof, but do not preclude the presence or addition of one or more other features, numerals, steps, operations, components, parts, or combinations thereof.

Unless otherwise defined, all terms used herein, including technical and scientific terms, have the same meaning as commonly understood by those skilled in the art to which the embodiments pertain. Terms, such as those defined in commonly used dictionaries, should be interpreted as having a meaning that is consistent with their meaning in the context of the relevant art, and will not be interpreted in an idealized or overly formal sense unless explicitly defined otherwise.

The present invention will be described in detail below.

To achieve the above objects, one aspect of the present invention provides a pharmaceutical composition for the prevention or treatment of neurofibromatosis type 1, comprising Raddeanin A, Polyphyllin B, or a pharmaceutically acceptable salt thereof as an active ingredient.

The “Raddeanin A” used in the present invention is an oleanane-type triterpenoid saponin compound primarily isolated from the rhizomes of Anemone raddeana. Structurally, it is in the form of a glycoside in which several sugar molecules are attached to a triterpene skeleton, and is known as a natural compound having effects on rheumatism, arthritis, and pain relief. However, there has been no report on the use of Raddeanin A for the prevention and treatment of neurofibromatosis type 1 through the inhibition of intracellular NF1-FBXW11 binding and the increase in NF1 protein.

The “Polyphyllin B” used in the present invention is a steroidal saponin-based compound primarily isolated from the rhizome of Paris polyphylla, also known as Chilyeobiljihwa or Jungru. It has a steroid glycoside structure with pennogenin as an aglycone, and is known as a natural compound effective for detoxification, anti-inflammation, and hemostasis. However, there has been no report on the use of Polyphyllin B for the prevention and treatment of neurofibromatosis type 1 through the inhibition of intracellular NF1-FBXW11 binding and the increase in NF1 protein.

The compounds of the present invention may be used in the form of a pharmaceutically acceptable salt, and include all salts, hydrates, and solvates prepared by conventional methods.

As the salt, acid addition salts formed by pharmaceutically acceptable free acids are useful. The acid addition salts are prepared by conventional methods, for example, by dissolving a compound in an excess amount of an aqueous acid solution and precipitating the salt using a water-miscible organic solvent, such as methanol, ethanol, acetone, or acetonitrile. Equimolar amounts of a compound and an acid in water or an alcohol (e.g., glycol monomethyl ether) may be heated, and then the mixture may be evaporated to dryness, or the precipitated salt may be filtered by suction. In this case, organic acids and inorganic acids may be used as the free acids; hydrochloric acid, phosphoric acid, sulfuric acid, nitric acid, tartaric acid, and the like may be used as the inorganic acids; methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycollic acid, gluconic acid, galacturonic acid, glutamic acid, glutaric acid, glucuronic acid, aspartic acid, ascorbic acid, carbonic acid, vanillic acid, hydroiodic acid, and the like may be used as the organic acids, but are not limited thereto.

Moreover, pharmaceutically acceptable metal salts may be prepared using bases. Alkali metal or alkaline earth metal salts may be obtained, for example, by dissolving a compound in an excess amount of an alkali metal hydroxide or alkaline earth metal hydroxide solution, filtering the undissolved compound salt, and then evaporating and drying the filtrate. In this case, as the metal salt, it is pharmaceutically suitable to prepare a sodium, potassium, or calcium salt, but is not limited thereto. Furthermore, a corresponding silver salt may be obtained by reacting an alkali metal or alkaline earth metal salt with a suitable silver salt (e.g., silver nitrate).

The pharmaceutically acceptable salt of the compound may include a salt of an acidic or basic group that may be present in the compound, unless otherwise indicated. For example, the pharmaceutically acceptable salt may include sodium, calcium, and potassium salts of a hydroxy group, and other pharmaceutically acceptable salts of an amino group include hydrobromide, sulfate, hydrogen sulfate, phosphate, hydrogen phosphate, dihydrogen phosphate, acetate, succinate, citrate, tartrate, lactate, mandelate, methanesulfonate (mesylate), and p-toluenesulfonate (tosylate) salts, and may be prepared through a method for preparing a salt known to those skilled in the art.

As used herein, the term “prevention” refers to all activities that inhibit neurofibromatosis type 1 or delay the onset thereof by Raddeanin A or Polyphyllin B of the present invention.

As used herein, the term “improvement” or “treatment” refers to all activities that improve or beneficially change parameters related to neurofibromatosis type 1 by Raddeanin A or Polyphyllin B of the present invention.

The pharmaceutical composition of the present invention may be in various oral or parenteral dosage forms. When formulating the composition, it may be prepared using one or more buffers (e.g., saline or PBS), antioxidants, bacteriostatic agents, chelating agents (e.g., EDTA or glutathione), fillers, extenders, binders, adjuvants (e.g., aluminum hydroxide), suspending agents, thickeners, wetting agents, disintegrants or surfactants, diluents, or excipients.

Solid preparations for oral administration include tablets, pills, powders, granules, capsules, and the like, and such solid preparations are prepared by mixing one or more compounds with at least one or more excipients, for example, starch (including corn starch, wheat starch, rice starch, potato starch, etc.), calcium carbonate, sucrose, lactose, dextrose, sorbitol, mannitol, xylitol, erythritol, maltitol, cellulose, methyl cellulose, sodium carboxymethyl cellulose, and hydroxypropyl methyl-cellulose, or gelatin. For example, tablets or dragees may be obtained by combining the active ingredient with a solid excipient, then pulverizing the same, and after adding suitable auxiliaries, processing the same into a granule mixture.

In addition to simple excipients, lubricants such as magnesium stearate and talc are also used. Liquid preparations for oral administration include suspensions, internal solutions, emulsions, or syrups, and may include various excipients such as wetting agents, sweeteners, aromatics, or preservatives, in addition to water and liquid paraffin, which are commonly used simple diluents. In some cases, cross-linked polyvinylpyrrolidone, agar, alginic acid, or sodium alginate may be added as a disintegrant, and may further include anti-agglutinants, fragrances, emulsifiers, solubilizers, dispersants, flavoring agents, antioxidants, packaging agents, pigments, and preservatives.

Preparations for parenteral administration include sterilized aqueous solutions, non-aqueous solutions, suspensions, emulsions, lyophilized preparations, or suppositories. As the non-aqueous solutions and suspensions, propylene glycol, polyethylene glycol, vegetable oils such as olive oil, and injectable esters such as ethyl oleate may be used. As a base for the suppositories, witepsol, macrogol, tween 61, cacao butter, laurin butter, glycerol, gelatin, and the like may be used.

The composition of the present invention may be administered orally or parenterally, and in the case of parenteral administration, it may be formulated in the form of an external preparation for skin; an injection; a transdermal preparation; or a nasal inhalant according to a method known to those skilled in the art.

In the case of the above injection, it must be sterilized and must be protected from contamination by microorganisms such as bacteria and fungi. In the case of injections, examples of suitable carriers include, but are not limited to, a solvent or dispersion medium containing water, ethanol, polyol (e.g., glycerol, propylene glycol, and liquid polyethylene glycol, etc.), mixtures thereof and/or vegetable oils. More preferably, suitable carriers may include Hanks' solution, Ringer's solution, phosphate buffered saline (PBS) containing triethanolamine, or isotonic solutions such as sterile water for injection, 10% ethanol, 40% propylene glycol, and 5% dextrose. To protect the injection from microbial contamination, various antibacterial and antifungal agents such as paraben, chlorobutanol, phenol, sorbic acid, and thimerosal may be further included. In addition, the injection may further include an isotonic agent such as sugar or sodium chloride in most cases.

In the case of inhalation preparations, the compound used according to the present invention may be conveniently delivered in the form of an aerosol spray from a pressurized pack or a nebulizer using a suitable propellant, for example, dichlorofluoromethane, trichlorofluoromethane, dichlorotetrafluoroethane, carbon dioxide, or other suitable gas. In the case of pressurized aerosols, the dosage unit may be determined by providing a valve that delivers a metered amount. For example, gelatin capsules and cartridges used in an inhaler or insufflator may be formulated to contain a powder mixture of the compound and a suitable powder base such as lactose or starch.

The composition of the present invention may be administered in a pharmaceutically effective amount. A pharmaceutically effective amount refers to an amount sufficient to treat a disease at a reasonable benefit/risk ratio applicable to medical treatment, and the effective dose level may be determined according to factors including the type and severity of the patient's disease, activity of the drug, sensitivity to the drug, administration time, administration route and excretion rate, treatment period, drugs used concurrently, and other factors well known in the medical field. The composition of the present invention may be administered as an individual therapeutic agent or in combination with other therapeutic agents, and may be administered sequentially or simultaneously with conventional therapeutic agents, and may be administered in a single or multiple doses. That is, the total effective amount of the composition of the present invention may be administered to a patient in a single dose, or may be administered by a fractionated treatment protocol in which multiple doses are administered over a long period of time. Considering all of the above factors, it is important to administer an amount that can achieve the maximum effect with a minimum amount without side effects, and this can be easily determined by those skilled in the art.

The dosage of the pharmaceutical composition of the present invention may vary in its range depending on the patient's body weight, age, sex, health status, diet, administration time, administration method, excretion rate, and severity of the disease. It may be increased or decreased according to the administration route, severity of obesity, sex, body weight, age, and the like.

The composition of the present invention may be used alone or in combination with methods using chemotherapy and biological response modifiers.

In one embodiment of the present invention, the “Raddeanin A” and “Polyphyllin B” may induce an increase in neurofibromin (NF1 protein).

Based on the observation that symptoms of NF1 haploinsufficiency develop due to a quantitative decrease in NF1 protein, the present inventors screened Raddeanin A and Polyphyllin B, which are compounds that induce an increase in NF1 protein, from about 2,000 types of natural compounds, and completed the present invention based on the fact that a pharmaceutical composition containing the same can be used as a therapeutic agent for neurofibromatosis type 1.

According to one embodiment of the present invention, “Raddeanin A” and “Polyphyllin B” may induce an increase in neurofibromin (NF1 protein) and may induce inhibition of the interaction or binding between neurofibromin and FBXW11 protein.

Based on the observation that symptoms of Nf1 haploinsufficiency develop due to a quantitative decrease in NF1 protein, the present inventors discovered FBXW11, which is involved in the stabilization of NF1 protein, using an F-box siRNA library, and confirmed that FBXW11 induces ubiquitination of NF1 by interacting with NF1 protein. Furthermore, the present inventors confirmed that Raddeanin A and Polyphyllin B induce inhibition of the interaction and binding between FBXW11 and NF1 protein, and completed the present invention based on the fact that a pharmaceutical composition containing the same can be used as a therapeutic agent for neurofibromatosis type 1.

Another aspect of the present invention provides a method for the prevention or treatment of neurofibromatosis type 1, comprising administering Raddeanin A, Polyphyllin B, or a pharmaceutically acceptable salt thereof to a subject.

The “Raddeanin A”, “Polyphyllin B”, “prevention”, and “treatment” are as described above.

In the present invention, the term “administration” refers to introducing a specific substance to a subject by an appropriate method.

As used herein the term “subject” refers to any animal, including humans, rats, mice, livestock, etc., intended for the treatment of a disease, preferably a mammal including humans, and more preferably a patient with neurofibromatosis type 1.

Still another aspect of the present invention provides a food composition for the prevention or improvement of neurofibromatosis type 1, comprising Raddeanin A, Polyphyllin B, or a pharmaceutically acceptable salt thereof as an active ingredient.

The “Raddeanin A”, “Polyphyllin B”, “prevention”, and “improvement” are as described above.

As used herein, the term “food” includes meat, sausage, bread, chocolate, candy, snacks, confectionery, pizza, ramen, other noodles, gum, dairy products including ice cream, various soups, beverages, teas, health drinks, alcoholic beverages, vitamin complexes, health functional foods, and health foods, and includes all foods in the conventional sense.

Since the food composition of the present invention can be consumed on a daily basis, it can be expected to have an effect of preventing or improving neurofibromatosis type 1, and thus can be very useful for the purpose of health promotion.

As used herein, the term “functional food” is synonymous with “food for special health use (FoSHU)” and refers to a food with high medicinal and medical effects that is processed to efficiently exhibit bioregulatory functions in addition to providing nutrients. Here, the term “functionality” means obtaining useful effects for health purposes, such as regulating nutrients for the structure and function of the human body or providing physiological actions. The food of the present invention can be manufactured by methods commonly used in the art, and during the manufacture, it can be manufactured by adding raw materials and ingredients commonly added in the art. In addition, the formulation of the food can also be manufactured without limitation as long as it is a formulation recognized as food. The food composition of the present invention may be prepared in various types of formulations. Unlike general medicines, it has the advantage of having no side effects that may occur during long-term administration of medicines because it uses food as a raw material, and has excellent portability. Therefore, the food of the present invention can be ingested as a supplement to enhance the effect of preventing or improving gastrointestinal diseases.

The term “health food” refers to food that has a more active health maintenance or promotion effect compared to general food, and the term “health supplement food” refers to food for the purpose of health supplementation. In some cases, the terms health functional food, health food, and health supplement food are used interchangeably.

Specifically, the health functional food may be a food prepared by adding the compound of the present invention to food materials such as beverages, teas, spices, gum, and confectionery, or by encapsulating, powdering, or suspending the same, which brings about specific health effects when consumed, but unlike general medicines, it has the advantage of having no side effects that may occur during long-term administration of medicines because it uses food as a raw material.

The food composition may further include a physiologically acceptable carrier, and the type of the carrier is not particularly limited and any carrier commonly used in the art may be used.

In addition, the food composition may include additional ingredients commonly used in food compositions to improve odor, taste, appearance, and the like. For example, it may include vitamins A, C, D, E, B1, B2, B6, and B12, niacin, biotin, folate, pantothenic acid, and the like. Additionally, it may include minerals such as zinc (Zn), iron (Fe), calcium (Ca), chromium (Cr), magnesium (Mg), manganese (Mn), and copper (Cu); and amino acids such as lysine, tryptophan, cysteine, and valine.

Moreover, the food composition may include food additives such as preservatives (potassium sorbate, sodium benzoate, salicylic acid, sodium dehydroacetate, etc.), disinfectants (bleaching powder and high-grade bleaching powder, sodium hypochlorite, etc.), antioxidants (butylated hydroxyanisole (BHA), butylated hydroxytoluene (BHT), etc.), coloring agents (tar color, etc.), coloring enhancers (sodium nitrite, sodium nitrate, etc.), bleaching agents (sodium sulfite), seasonings (MSG sodium glutamate, etc.), sweeteners (dulcin, cyclamate, saccharin, sodium, etc.), flavorings (vanillin, lactones, etc.), leavening agents (alum, potassium D-bitartrate, etc.), strengthening agents, emulsifiers, thickeners (pastes), coating agents, gum bases, anti-foaming agents, solvents, and improvers. The additives may be selected according to the type of food and used in an appropriate amount.

As an example, the food composition of the present invention may be used as a health beverage composition, and in this case, it may contain various flavoring agents or natural carbohydrates as additional ingredients like conventional beverages. The above-mentioned natural carbohydrates may be monosaccharides such as glucose and fructose; disaccharides such as maltose and sucrose; polysaccharides such as dextrin and cyclodextrin; and sugar alcohols such as xylitol, sorbitol, and erythritol. Examples of the sweeteners may include natural sweeteners such as thaumatin and stevia extract, and synthetic sweeteners such as saccharin and aspartame. The ratio of the natural carbohydrate may generally be about 0.01 to 0.04 g, specifically about 0.02 to 0.03 g per 100 mL of the health beverage composition of the present invention.

Additionally, the health beverage composition may contain various nutrients, vitamins, electrolytes, flavoring agents, coloring agents, pectic acid, salts of pectic acid, alginic acid, salts of alginic acid, organic acids, protective colloidal thickeners, pH adjusters, stabilizers, preservatives, glycerin, alcohol, or carbonating agents. Besides, it may contain fruit flesh for the production of natural fruit juice, fruit juice beverages, or vegetable beverages. These ingredients may be used independently or in combination. The ratio of these additives is not critically important, but it is generally selected in the range of 0.01 to 0.1 parts by weight per 100 parts by weight of the health beverage composition of the present invention.

The composition of the present invention for the treatment of neurofibromatosis type 1, comprising Raddeanin A or Polyphyllin B, can inhibit FBXW11, which induces the degradation of neurofibromin (NF1 protein), and induces an increase in NF1 protein by inhibiting the interaction between FBXW11 and neurofibromin, thereby being useful for the treatment of neurofibromatosis type 1.

### BRIEF DESCRIPTION OF THE DRAWINGS

The above and other features and advantages of the present invention will become more apparent by describing in detail exemplary embodiments thereof with reference to the attached drawings in which:

FIG. 1 shows the results of measuring the expression levels of neurofibromin (NF1 protein) and pERK1/2 after transfecting each F-box protein-specific siRNA into human fibroblasts using an F-box siRNA library;

FIG. 2 shows the results of measuring the expression levels of neurofibromin (NF1 protein) and pERK1/2 after transfecting FBXO3 and FBXW11 protein-specific siRNAs into Hela cells. GAPDH was used as a control;

FIG. 3 shows the results of evaluating the stability of neurofibromin protein using cycloheximide (CHX) chase analysis in Hela cells;

FIG. 4 shows the results of evaluating the stability of neurofibromin protein using cycloheximide (CHX) chase analysis after transfecting FBXW11 siRNA, FBXO3 siRNA, or control siRNA into Hela cells;

FIG. 5 shows the results of evaluating the amount of neurofibromin protein after transfecting a vector for expressing FBXW11 or FBXO3 cDNA into Hela cells;

FIG. 6 shows the results of Western blot analysis of neurofibromin and pERK1/2 levels after treating Nf1 haploinsufficient mouse embryonic fibroblasts (Nf1^{+/-}MEF) with PDTC, an FBXW11 inhibitor, or BC-1215, an FBXO3 inhibitor;

FIG. 7 shows the degree of binding of FBXW11 to each domain after dividing the NF1 protein into six domains (D1-D5, where D3 is divided into isoform 1/GRD1 and isoform 2/GRD2) and attaching a GFP tag to each;

FIG. 8 shows the degree of binding of Flag-labeled FBXW11 and FBXO3 to isoform 1/GRD1 of the NF1 protein using a co-immunoprecipitation (Co-IP) experiment;

FIG. 9 shows the results of evaluating the direct physical interaction between FBXW11 and GRD1 in cells using a NanoBiT assay;

FIG. 10 shows the results of confirming whether a direct SCF E3 ubiquitin ligase-substrate interaction exists between FBXW11 and neurofibromin according to changes in the amount of SCF^{FBXW11} using a TUBEs assay;

FIG. 11 shows the results of comparing the amount of ubiquitinated neurofibromin among proteins isolated after treatment with FBXW11 siRNA or control siRNA using a TUBEs assay by Western blot;

FIG. 12 shows the behavioral experimental procedures for wild-type (WT, Nf1^{+/+}; FBXW11^{+/+}), Nf1^{+/-}; FBXW11^{+/+} (NF1), Nf1^{+/+}; FBXW11+/− (FBX), and double heterozygous male mice (Nf1^{+/-}; FBXW11+/−) in which both the Nf1 gene and the FBXW11 gene are haploinsufficient;

FIG. 13 shows the results of evaluating the performance of the open field (OF) task in the Nf1^{+/-}; FBXW11^{+/-} mouse model and control mouse models;

FIG. 14 shows the results of evaluating the performance of the delay discounting Task (DDT) in the Nf1^{+/-}; FBXW11^{+/-} mouse model and control mouse models;

FIG. 15 shows the results of evaluating the performance of the social preference (SP) task in the Nf1^{+/-}; FBXW11^{+/-} mouse model and control mouse models;

FIG. 16 shows the results of evaluating the performance of the cliff avoidance reaction (CAR) task in Nf1^{+/-}; FBXW11^{+/-} mouse model and control mouse models;

FIG. 17 shows the results of measuring the number of Nf1 and pERK1/2 immunoreactive cells in the brain tissue of Nf1^{+/-}; FBXW11^{+/-} mouse model and control mouse models through immunohistochemical (IHC) analysis;

FIG. 18 shows the results of measuring NF1-GRD1 protein using the Nano-Glo Hibit lytic assay for HEK293T stable cells expressing the NF1-GRD1 protein;

FIG. 19 shows the results of primarily screening 46 substances with activity more than twice as high as that of a control group after treating HEK293T cells with each of approximately 2,000 types of natural compounds at concentrations of 1, 2.5, 5, and 10 μM for 24 hours;

FIG. 20 shows the structures of the pFN33KLgBit-TK-neo Flexi vector and the pFN35KSmBit-TK-neo Flexi vector constructed to measure the mutual binding of FBXW11-GRD1;

FIG. 21 shows the results of measuring the NF1-FBXW11 interaction through NanoBit protein interaction analysis for each of the 46 natural compounds that induce an increase in neurofibromin, which were selected through the primary screening; and

FIG. 22 shows the treatment mechanisms for neurofibromatosis type 1 of Raddeanin A and Polyphyllin B, which were finally selected through natural product screening.

### DETAILED DESCRIPTION OF THE INVENTION

Hereinafter, the present invention will be described in more detail with reference to the following examples. The matters described in the following examples are preferred embodiments of the present invention, and the scope of the present invention is not limited by the matters described in these examples.

### Example 1: Screening for Proteins Mediating Neurofibromin Degradation

1-1. Screening for Proteins Mediating Neurofibromin Degradation Using F-box siRNA Library

Cultured human diploid fibroblasts were collected, and for siRNA screening, cell suspensions were incubated in complete medium (without antibiotics) at 37 °C in polypropylene tubes (<1 h) until used in the chemical reverse transfection in 6-well plates. To transfect each siRNA into the cultured cells, transfection complexes were prepared in Opti-MEM serum-free medium (Invitrogen) by mixing 0.3 μL of siPORT NeoFX Transfection Reagent (Ambion) and 10 nM of siRNA (individual siRNA members of the F-boxes, Ambion).

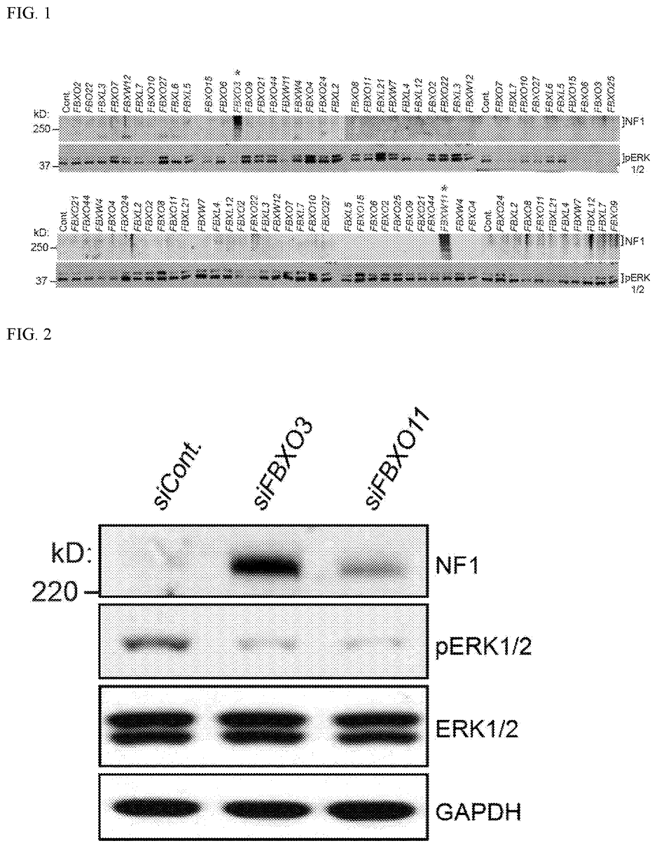

Human fibroblast cells (2×10⁵ cells/well) and Hela cells were plated in a 6-well format simultaneously with the addition of transfection complexes, and after incubating the cells for 72 hours, the levels of neurofibromin (NF1) protein and the phosphorylation of ERK1/2(pERK1/2 ), a downstream factor of the Ras signaling pathway, were analyzed by Western blotting, and the results are shown in FIG. 1 and FIG. 2.

As shown in FIGS. 1 and 2, it was confirmed that neurofibromin protein was significantly accumulated when siRNA targeting FBXW11 (BTRC2) or FBXO3 among various F-box siRNAs was treated (FIG. 1), and it was further confirmed that FBXW11 or FBXO3 siRNA treatment not only stabilized neurofibromin protein but also inhibited the level of pERK1/2, a downstream factor of the Ras signaling pathway (FIG. 2).

1-2. Demonstration of Increase in NF1 Protein LEVEL and Normalization of Ras Signaling Using FBXW11 and FBXO3 siRNA
A. Evaluation of Protein Stability through Cycloheximide Chase Analysis

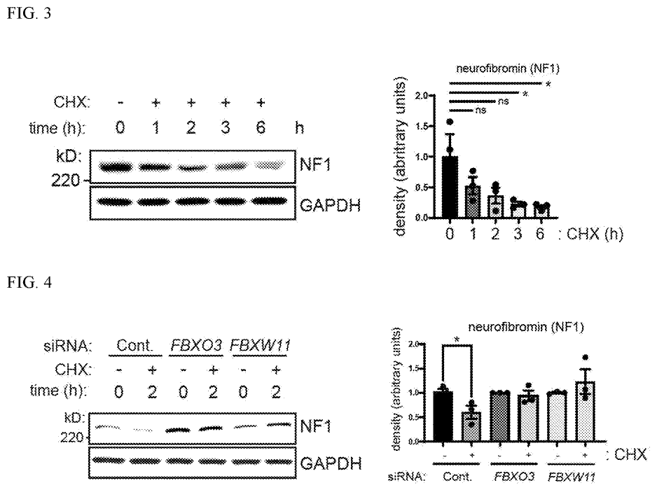

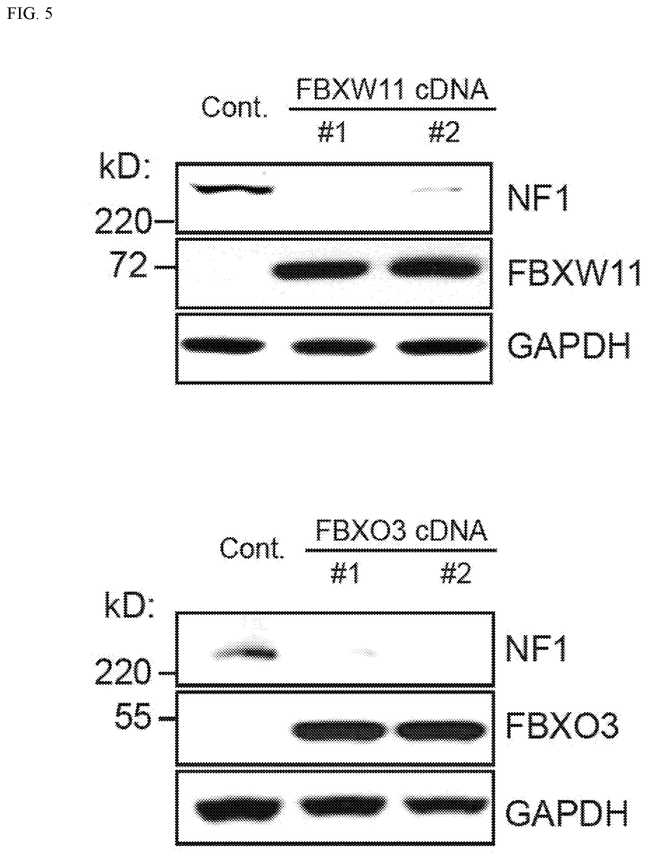

The stability of neurofibromin protein was evaluated using cycloheximide (CHX) chase analysis in Hela cells. Hela cells were transfected with FBXW11 siRNA, FBXO3 siRNA, or control siRNA in the same manner as described above. After 48 to 72 hours of transfection, the cells were treated with 20 μg/mL of CHX (Sigma-Aldrich), and the results of confirming the change in the amount of neurofibromin protein over time by Western blot are shown in FIG. 3 and FIG. 4.

As shown in FIGS. 3 and 4, in the control cells, the expression level of neurofibromin decreased 2 hours after treatment with CHX to inhibit protein synthesis (FIG. 3). On the other hand, in Hela cells transfected with FBXW11 or FBXO3 siRNA, the level of neurofibromin did not change even 2 hours after CHX treatment (FIG. 4).

These results confirmed that inhibiting the expression of FBXW11 or FBXO3 increases the stability of the neurofibromin protein.

### B. Evaluation of the Effects of FBXW11 and FBXO3 Overexpression

Hela cells were transfected with vectors expressing FBXW11 or FBXO3 cDNA using polyethylenimine (PEI), and the results of confirming the change in the amount of endogenous neurofibromin after 48 hours by Western blot are shown in FIG. 5.

As shown in FIG. 5, similar results were observed in experiments where FBXW11 or FBXO3 was overexpressed by transfection, and it was confirmed that the expression level of endogenous neurofibromin decreased compared to the control group.

### C. Evaluation of the Effects of FBXW11 and FBXO3 Inhibitor Treatment

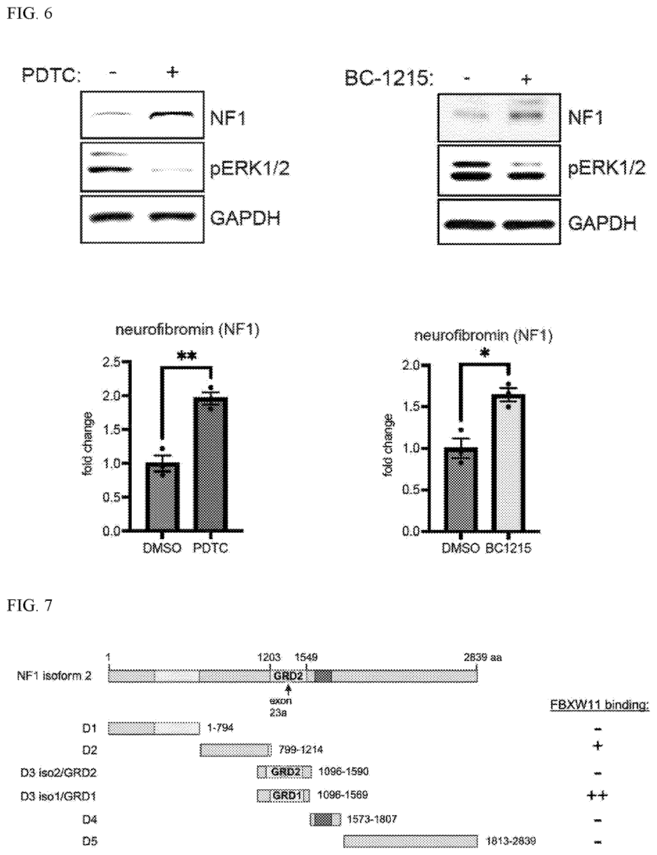

Neurofibromin haploinsufficient murine embryonic fibroblasts (Nf1^{+/-} MEFs) were treated with PDTC (50 μM), an FBXW11 inhibitor, or BC-1215 (20 μg/mL), an FBXO3 inhibitor, for 6 hours, and the levels of neurofibromin and pERK1/2 were analyzed by Western blot, and the results are shown in FIG. 6.

As shown in FIG. 6, it was confirmed that even in murine embryonic fibroblasts in which one of the FBXW11 or FBXO3 genes was deleted (Nf1^{+/-} MEFs), the expression level of neurofibromin increased and the level of pERK1/2 decreased.

Taken together, these results suggest that FBXW11 and FBXO3 mediate the degradation of Neurofibromin.

### 1-3. 1-3. Demonstration of Direct Interaction Between SCF^{FBXW11} Complex and Neurofibromin and Ubiquitin-Proteasome Degradation Pathway

### A. Identification of Binding Domains Between FBXW11 and Neurofibromin Using Co-immunoprecipitation (Co-IP)

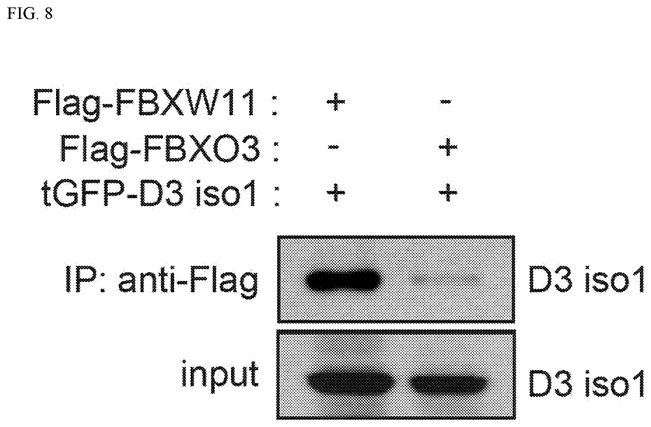

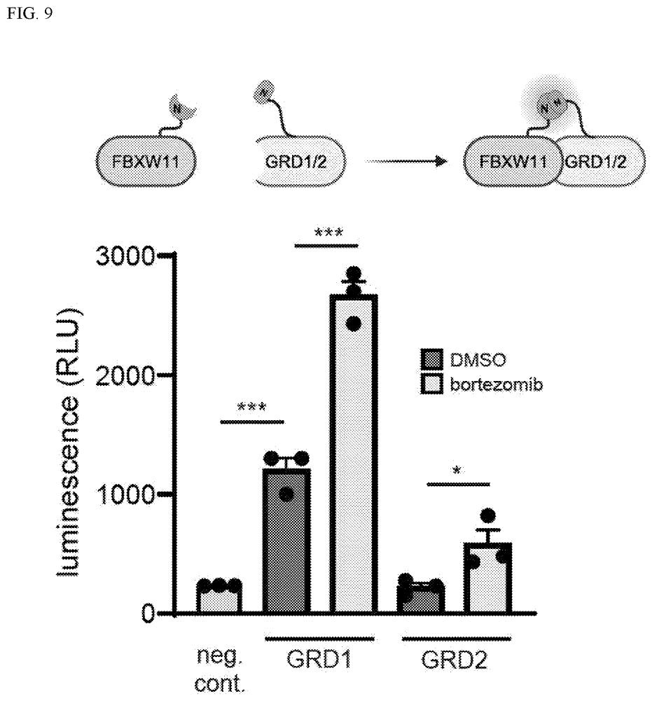

Expression vectors were constructed by dividing the neurofibromin protein into six domains (D1-D5, where D3 was subdivided into isoform 1/GRD1 and isoform 2/GRD2) and attaching a GFP tag to each. Flag-tagged FBXW11 and each GFP-neurofibromin domain vector were co-transfected into HEK293T cells, and the cells were treated with the proteasome inhibitor MG132 (15 μM) 6 hours before cell harvesting. After cell lysis, immunoprecipitation was performed using an anti-Flag antibody, and the results of detecting the co-precipitated GFP-neurofibromin domains by Western blot using an anti-GFP antibody are shown in FIG. 7 and FIG. 8.

As shown in FIG. 7, two isoforms resulting from alternative splicing of exon 23a within the GAP-related domain (GRD) of neurofibromin were generated. It was confirmed that isoform 2 includes exon 23a in the GRD (D3 iso2/GRD2), while isoform 1 lacks exon 23a (D3 iso1/GRD1).

Furthermore, as shown in FIG. 8, co-immunoprecipitation experiments confirmed that Flag-tagged FBXW11 binds to the D3 domain containing the GRD among several domains of neurofibromin, and in particular, it binds more strongly to isoform 1 (D3 iso1/GRD1), which is predominantly expressed in the brain and has high Ras-GAP activity.

### B. Confirmation of Interaction Between FBXW11 and Neurofibromin GAP-Related Domain (GRD) 1 Using NanoBiT Assay

To assess the direct physical interaction between FBXW11 and GRD1 in live cells, a Nanoluciferase-based complementation assay (NanoLuc Binary Technology, NanoBiT) and plasmids encoding GRD1, GRD2, or FBXW11 were used.

Plasmids were constructed by splitting the NanoLuciferase enzyme into a large fragment (LgBiT) and a small fragment (SmBiT), which were then fused to FBXW11 and the neurofibromin GRD1/GRD2 domains, respectively. The fusion plasmids were co-transfected into HEK 293T cells, and after 48 hours, the luciferase activity (bioluminescence signal) restored by protein interaction was measured by adding substrate to the live cells; the results of simultaneously confirming the effect of treatment with the proteasome inhibitor bortezomib (1 μM) are shown in FIG. 9.

As shown in FIG. 9, the interaction between FBXW11 and GRD1 significantly increased compared to the control, and it was confirmed that this interaction was further augmented by treatment with the proteasome inhibitor bortezomib.

### C. Determination of Ubiquitination of Neurofibromin

To determine whether a direct SCF E3 ubiquitin ligase-substrate interaction existed between FBXW11 and neurofibromin, in vitro ubiquitination analysis was performed using an in vitro ubiquitination assay and a tandem ubiquitin-binding entities (TUBEs) assay.

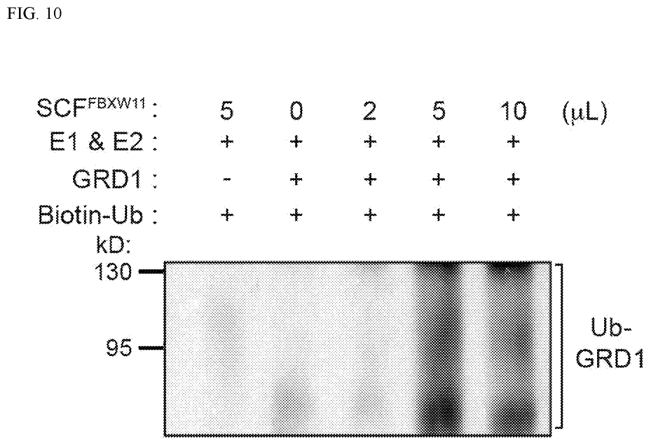

First, the SCF^{FBXW11} complex (E3) was purified through immunoprecipitation after co-transfecting HEK293T cells with Flag-FBXW11, CUL1, SKP1, and RBX1. The purified SCF^{FBXW11} complex, purified GFP-GRD1 (substrate), E1 activating enzyme, E2 activating enzyme (UbcH3/Cdc34), ubiquitin, and ATP were reacted together. After the reaction, the ubiquitination of GRD1 was confirmed by Western blot using an anti-GFP antibody, and the results are shown in FIG. 10.

As shown in FIG. 10, as a result of confirming the ubiquitination of neurofibromin using an in vitro ubiquitination assay, a GRD1-associated ubiquitin smear was observed as the level of SCF^{FBXW11} increased, whereas it was not detectable in the absence of FBXW11 or GRD1.

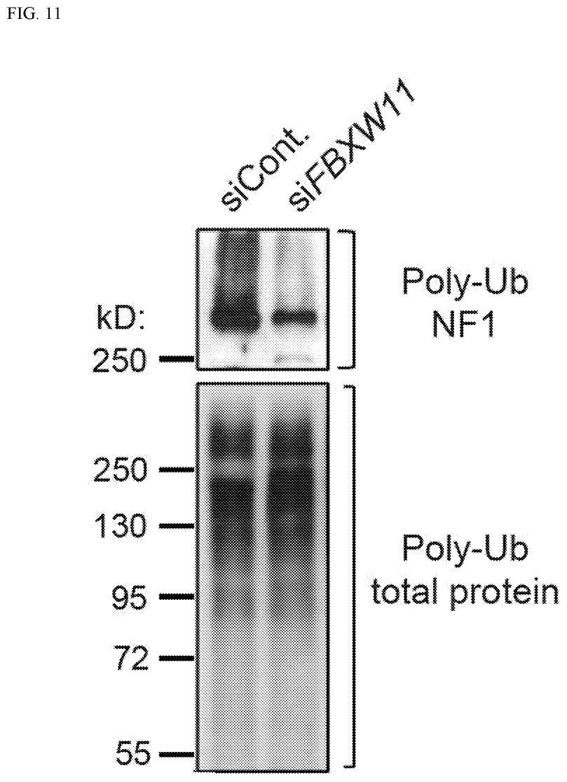

In addition, HEK293T cells were treated with either FBXW11 siRNA or control siRNA. Ubiquitinated proteins were isolated from the cell lysates using TUBE (Tandem Ubiquitin Binding Entities) resin, which binds to ubiquitinated proteins. Subsequently, the amount of ubiquitinated neurofibromin among the isolated proteins was compared by Western blot, and the results are shown in FIG. 11.

As shown in FIG. 11, western blot analysis following affinity purification with TUBES-agarose revealed a substantial decrease in ubiquitin-conjugated neurofibromin in cells treated with FBXW11 siRNA compared to the control.

Therefore, it was confirmed that the SCF^{FBXW11} complex could mediate the ubiquitination and subsequent proteasomal degradation of neurofibromin.

### Example 2: Demonstration of the Efficacy of Target Gene FBXW11 for the Development of NF1 Therapeutics In Vivo

2-1. Construction of Nf1^{+/-}; FBXW11^{+/-} Mouse Models

To test whether disruption of FBXW11 could restore neurofibromin levels and ameliorate the neurodevelopmental phenotypes associated with Nf1 haploinsufficiency, we generated a genetically engineered mouse model (GEMM) deficient in FBXW11.

By crossing Nf1 haploinsufficient mice (Nf1^{+/-}) and FBXW11 haploinsufficient mice (FBXW11^{+/-}), double heterozygous male mice with haploinsufficiency of both genes (Nf1^{+/-}; FBXW11^{+/-}) were generated on an inbred C57BL6J strain background.

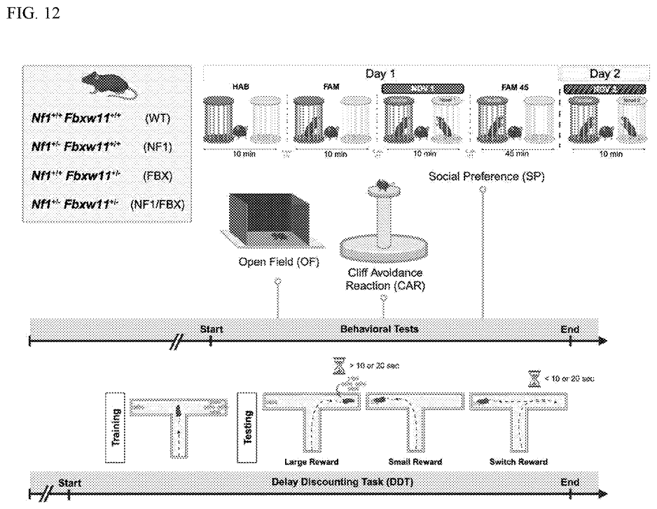

Wild-type (WT, Nf1^{+/+}FBXW11^{+/+}), Nf1^{+/-}, FBXW11^{+/+} (NF1), and Nf1^{+/+}; FBXW11^{+/-} (FBX) mice were used as controls. The behavioral experimental procedures for the experimental and control groups of mice generated in this manner are shown in FIG. 12.

### 2-2. Behavioral Experiments

### A. Open Field (OF) Task

Mice were placed in the dark in an open chamber (40×40 cm, 30 cm height) for 60 minutes, and the total distance traveled (m) was recorded using AnyMaze software. Behavior was evaluated not only for the entire duration (60 minutes) but also in 10-minute increments. Furthermore, to ensure that the experimenters were blind to the genotype, letter codes were assigned to the video files before scoring the behavior.

### B. Delay Discounting Task (DDT)

Mice were trained to identify which side of a T maze (each arm measured 36 cm length, 8 cm width, and 12.5 cm height) had a large reward (4 Cocoa Krispies) compared to a small reward (1 Cocoa Krispie). Training was conducted daily with 6 trials per day without delay, until each mouse correctly found the large reward at least 10 out of 12 times over two consecutive days. After this, the delay was initiated, and testing was conducted for a total of 6 days with 6 trials per day, with the first two days designated as “teaching trials.” During this period, the mouse was allowed to move freely to the side with the small reward, whereas if they moved to the side with the large reward, they were blocked in that arm for a 10-second delay, then the large reward was given. During the subsequent 4-day testing period, the mice were not blocked even if they moved toward the large reward, and the reward was provided only if they waited on their own. Experiments were conducted in this manner, and the analysis was performed based on 12 trials during the final two days. The mice were food-restricted during this time to increase food motivation, with daily measurement of weights and careful inspection of the health of each mouse.

### C. Social Preference (SP) Task

To evaluate autism spectrum disorder (ASD)-like behaviors, a social preference (SP) task was performed.

Specifically, the three-chamber social preference task occurs over two days. After being single-housed for at least 1 week, testing begins on Day 1 when the mouse is placed in the center of the three-chamber apparatus with two empty wire-cups for 10 minutes of habituation (HAB). After habituation, the mouse is blocked using clear plexiglass in the center of the apparatus during a 5-minute inter-trial interval (ITI). The examiner then places a partner mouse under one of the wire cups before allowing the test mouse to investigate the partner mouse for 10 minutes of familiarization (FAM). During the next ITI, another partner mouse (novel 1) is placed under the second cup, and for 10 minutes (NOV 1), the test mouse can investigate the familiar mouse and the novel 1 mouse. The examiner removes the novel 1 mouse during the ITI, and to ensure familiarity, the test mouse then spends 45 minutes with the familiar mouse (FAM45) under the cup. 24 hours later, the same familiar mouse is placed under the same wire cup, and a new novel mouse (novel 2) is placed under the other wire cup. The test mouse is introduced to the apparatus and given 10 minutes to investigate (NOV 2). Time spent near each wire cup is recorded and scored with the automated AnyMaze software during the November 1 and November 2 time blocks.

D. Cliff Avoidance Reaction (car) Task

A round, plastic platform (diameter 20 cm, thickness 2 cm) supported by a plastic rod (height 50 cm) was used to assess Cliff Avoidance Reaction (CAR). The platform was stabilized in a kiddie pool with a rubber bottom to provide a cushion in case the animal fell. Mice were placed in the center of the platform, and their behavior was recorded for 60 minutes. If a mouse fell from the platform, it was immediately placed back on the platform, and the test continued for up to 60 minutes. A mouse was considered to have a normal Cliff Avoidance Reaction if it did not fall off the platform. CAR was calculated as the percentage of mice that demonstrated a normal reaction in each group. The time spent on the platform was recorded and scored with the automated AnyMaze software. The “edge area” was defined by outlining the outer 1 inch of the round platform with one inner circle and one outer circle in the AnyMaze software, and the “over the edge zone” was defined by outlining an outer circle that was 2 inches from the edge of the platform.

### 2-3. Results of the Behavioral Experiments

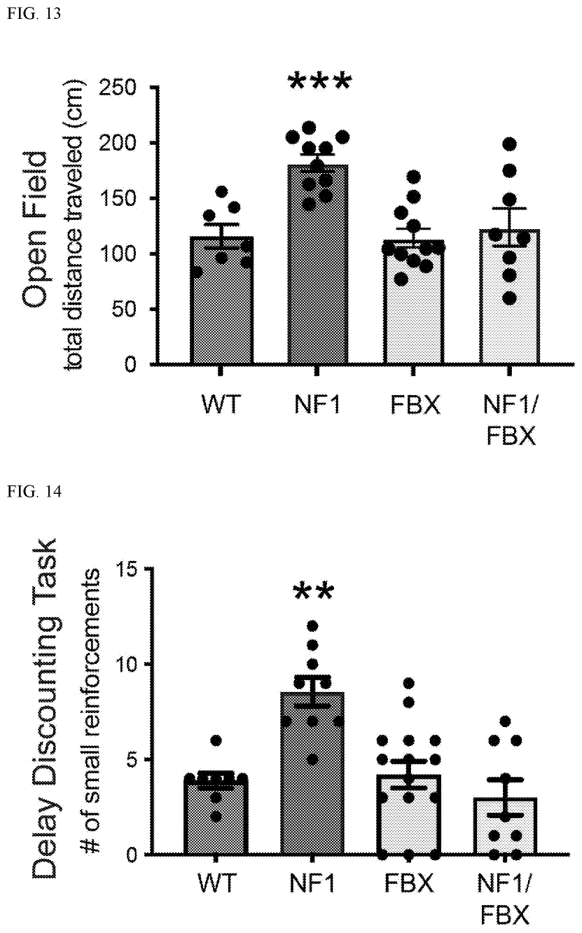

To evaluate whether the disruption of FBXW11 could restore neurofibromin levels and ameliorate the neurodevelopmental phenotypes associated with Nf1 haploinsufficiency, the neurocognitive task performance, as described in Example 2-2, was assessed in the Nf1^{+/-}; FBXW11^{+/-} mouse model, and the results are shown in FIGS. 13 to 16.

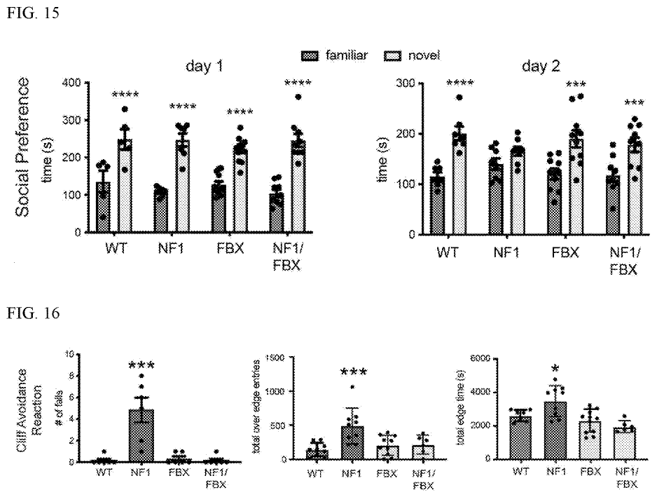

As shown in FIGS. 13 to 16, Nf1^{+/-} mice that were wild type for FBXW11 (NF1) showed increased hyperactivity in the OF task measured by total distance traveled (FIG. 13), chose a higher percentage of small rewards rather than waiting to receive the large reward (FIG. 14), demonstrated decreased distinction between a novel and a familiar partner mouse on Day 2 of the social preference (SP) task (FIG. 15), and had an increase in the number of falls, over-edge time, and edge time while performing the CAR task (FIG. 16). However, FBXW11 haploinsufficiency rescued all these behavioral deficits, and as a result, no significant differences were observed between control wild-type (WT) and Nf1^{+/-}; FBXW11^{+/-} mice (NF1/FBX) in the performance of these tasks.

2-4. Immunohistochemical (ihc) Analysis of Brain Tissue

Mice were perfused transcardially with phosphate-buffered saline (PBS), followed by 4% paraformaldehyde, and the brains were removed and coronally sectioned at 35 μm. After washes with PBS, H₂O₂, and phosphate-buffered saline Triton X-100 (PBST), the primary antibody diluted in PBST was applied to the sections overnight. The next day, after washing three times with PBS, the sections were placed in secondary antibody diluted in PBST for 90 minutes, then amplified by ABC (1:500, Vector Labs) for 90 minutes. After washing, vector SG was used to identify immunoreactivity (Vector Labs). The sections were stored in PB and then mounted on slides using a mounting medium containing DAPI (Vectashield). Photomicrographs were taken at 200× magnification, and neurofibromin-immunoreactive (NF1-ir) cells and pERK-immunoreactive (pERK-ir) cells were counted by a blind observer using ImageJ software. The antibodies used to identify each protein are as follows:
Neurofibromin: Primary antibody: rabbit anti-neurofibromin polyclonal antibody (1:400, LS-B8110); Secondary antibody: biotinylated anti-rabbit antibody (1:200, BA-1000).
pERK: Primary antibody: rabbit anti-pERK polyclonal antibody (1:200, Cell Signaling Technology); Secondary antibody: biotinylated anti-rabbit antibody (1:200, BA-1000).
GFP: Primary antibody: mouse anti-GFP polyclonal antibody (1:100, Life Science Technology); Secondary antibody: anti-mouse AlexaFluor488 (1:200, Life Sciences Technology).

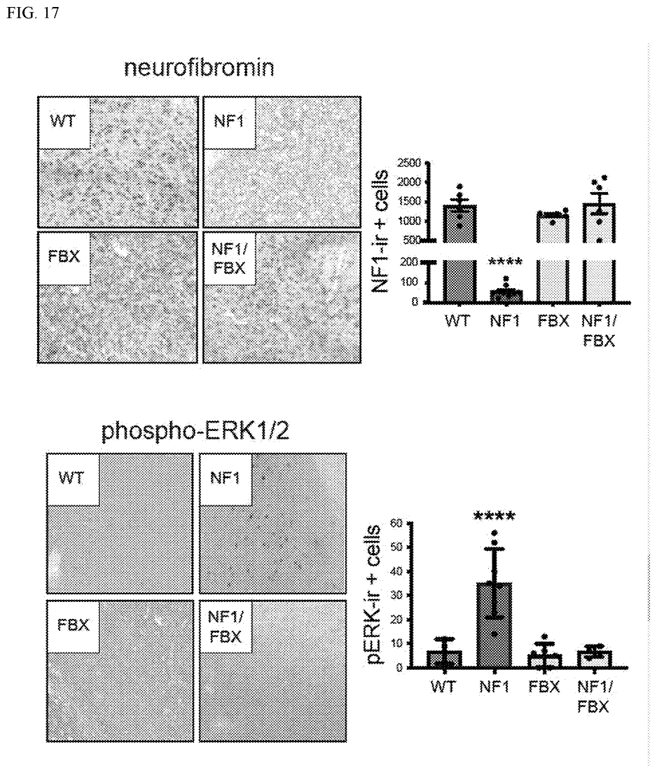

The results of measuring the number of neurofibromin-and pERK-immunoreactive cells through IHC staining of the mouse brain tissue as described above are shown in FIG. 17.

As shown in FIG. 17, immunohistochemical (IHC) analysis confirmed that the expression levels of neurofibromin protein were significantly increased, while the expression levels of pERK1/2 were decreased, in the brain tissue of Nf1^{+/-}; FBXW11^{+/-} mice compared to Nf1^{+/-} mice.

In summary, these results confirmed that FBXW11 haploinsufficiency increases neurofibromin levels, suppresses ERK activation, and rescues the neurodevelopmental deficits observed in Nf1^{+/-} mice.

### Example 3: Screening of Substances for the Treatment of Neurofibromatosis Type 1

### 3-1. Establishment of NF1-GRD1 Expressing Cell Lines

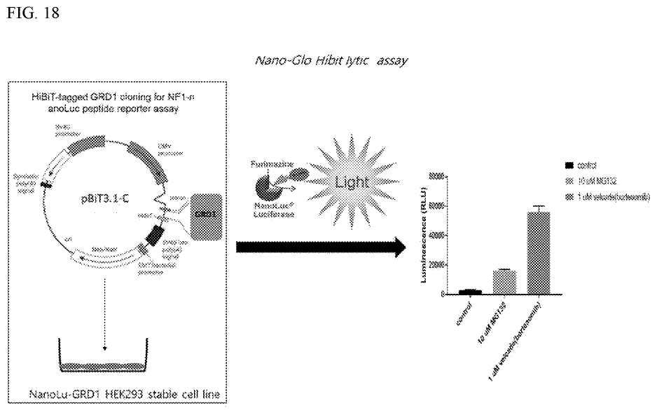

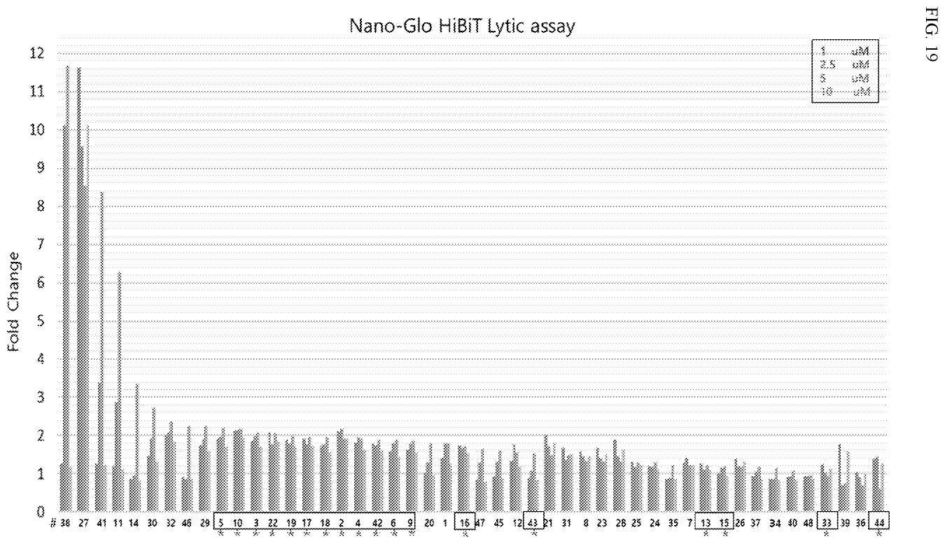

To produce a stable cell line expressing the NF1-GRD1 protein, NF1-GRD1 was cloned into a pBiT3.1-C vector combined with a 19 kDa Nanoluciferase reporter. This vector was then transfected into HEK293T cells to establish a stable cell line that consistently expresses the NF1-GRD1 protein. Subsequently, the cells were treated with Nano-Glo HiBiT Lytic Reagent (LgBiT+Furimazine+Lysis Buffer) to measure the luminescence resulting from the production of NanoLuc Luciferase and its reaction with the substrate, and the results are shown in FIG. 18.

As shown in FIG. 18, in the untreated control group, low activity was measured due to the degradation of NF1-GRD1 protein via proteasomes; however, upon treatment with protease inhibitors MG132 and Velcade (bortezomib), higher luminescence was measured compared to the control group. These results confirmed that the cell line of the present invention stably expresses NF1-GRD1.

### 3-2. Primary Screening for the Discovery of Natural Products Inducing Quantitative Increase in Intracellular Neurofibromin

Subsequently, each of the approximately 2,000 natural compounds obtained from the National Institute of Korean Medicine Development (NIKOM) and the Korea Chemical Bank was treated on the cells at concentrations of 1, 2.5, 5, and 10 μM for 24 hours. The quantification of NF1-GRD1 was then measured by luciferase activity using Nano-Glo (Promega) reagents. Forty-six substances that exhibited activity more than twofold higher than that of the control group were primarily selected, and the results are shown in FIG. 19.

### 3-3. Secondary Screening for the Discovery of Natural Products Inhibiting NF1-FBXW11 Interactions and Binding

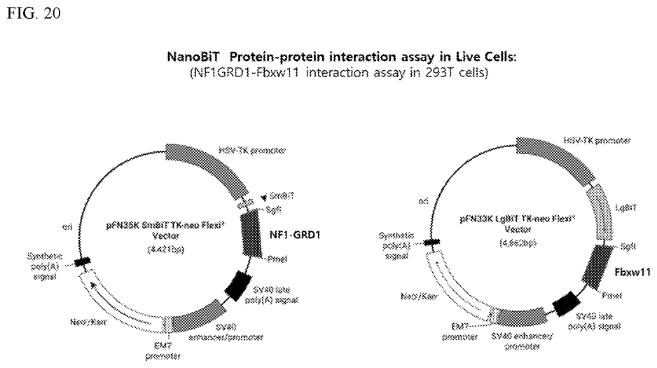

The interaction between FBXW11 and NF1-GRD1 was examined using the pFN33KLgBit-TK-neo Flexi and pFN35KSmBit-TK-neo Flexi vectors of the NanoBiT system (Promega). In this system, FBXW11 was inserted into the pFN33KLgBit-TK-neo Flexi vector, which expresses a 17.6 kDa largeBiT, and NF1-GRD1 was inserted into the pFN35KSmBit-TK-neo Flexi vector, which expresses a smallBiT consisting of 11 amino acids; the structures of each Flexi vector are shown in FIG. 20.

Subsequently, HEK 293T cells cultured in 96-well plates were transfected with the NanoBiT fusion GRD1 and FBXW11 plasmids using FuGENE HD Transfection Reagent (Promega).

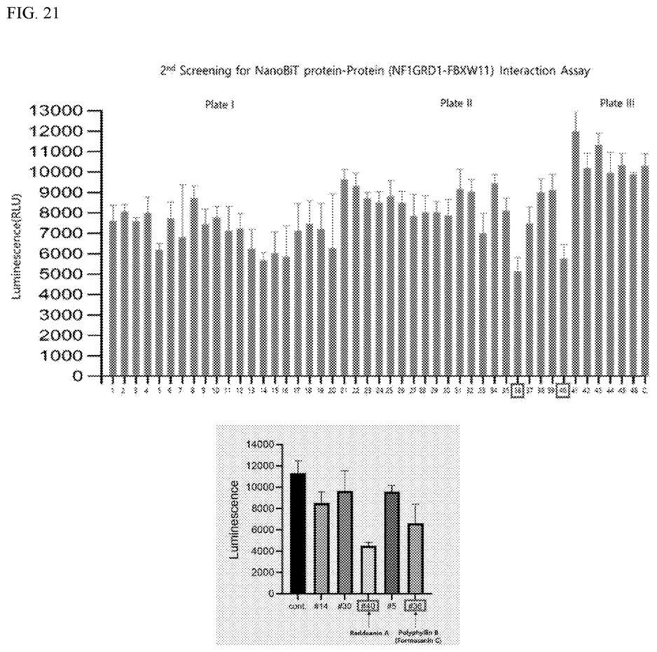

After 48 hours, the cells were treated for 2 hours with each of the 46 natural compounds selected through the primary screening that induce an increase in neurofibromin. Nano-Glo Live Cell Substrate (Promega) was then reacted in each well for 5 minutes, after which the luciferase activity of each well was measured using a Glo-MAX luminometer (Promega), and the results are shown in FIG. 21.

As shown in FIG. 21, the measurement of luciferase activity in each well revealed that Raddeanin A (#40) and Polyphyllin B (#36) exhibited lower luciferase activity than other compounds. These results confirmed that these compounds inhibit the interaction and binding of NF1-FBXW11 and induce a quantitative increase in NF1.

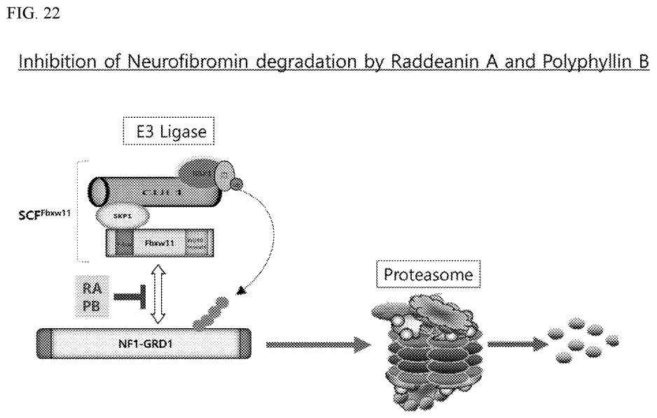

Accordingly, as shown in FIG. 22, Raddeanin A and Polyphyllin B of the present invention have the efficacy to induce an increase in NF1 protein by inducing inhibition of FBXW11 and inhibiting the mutual binding of FBXW11 and NF1, and thus it was confirmed that they can be effectively used for the treatment of Neurofibromatosis type 1.

While the invention has been shown and described with reference to certain preferred embodiments thereof, it will be understood by those skilled in the art that various changes in form and details may be made therein without departing from the spirit and scope of the invention as defined by the appended claims. Therefore, the scope of the invention is defined not by the detailed description of the invention but by the appended claims, and all differences within the scope will be construed as being included in the present invention.

What is claimed is:

1. A pharmaceutical composition for the prevention or treatment of neurofibromatosis type 1, comprising Raddeanin A, Polyphyllin B, or a pharmaceutically acceptable salt thereof as an active ingredient.

2. The pharmaceutical composition for the prevention or treatment of neurofibromatosis type 1 according to claim 1, wherein the Raddeanin A or Polyphyllin B induces an increase in neurofibromin (NF1) protein.

3. The pharmaceutical composition for the prevention or treatment of neurofibromatosis type 1 according to claim 1, wherein the Raddeanin A or Polyphyllin B induces inhibition of FBXW11 protein.

4. The pharmaceutical composition for the prevention or treatment of neurofibromatosis type 1 according to claim 1, wherein the Raddeanin A or Polyphyllin B induces inhibition of the interaction or binding between NF1 protein and FBXW11 protein.

5. A method for the prevention or treatment of neurofibromatosis type 1, comprising administering Raddeanin A, Polyphyllin B, or a pharmaceutically acceptable salt thereof to a subject.

6. The method for the prevention or treatment of neurofibromatosis type 1 according to claim 4, wherein the subject is a patient with neurofibromatosis type 1.

7. A food composition for the prevention or improvement of neurofibromatosis type 1, comprising Raddeanin A, Polyphyllin B, or a pharmaceutically acceptable salt thereof as an active ingredient.
